(19) United States
(12) Patent Application Publication
(10) Pub. No.: US 2023/0187017 A1
(43) Pub. Date: Jun. 15, 2023

(54) THREE-DIMENSIONAL ELECTRON DENSITY MAP SPECIFYING APPARATUS, SYSTEM, METHOD, AND PROGRAM

(71) Applicant: RIGAKU CORPORATION, Tokyo (JP)

(72) Inventors: Takashi SATO, Tokyo (JP); Takashi MATSUMOTO, Tokyo (JP); Tomokazu HASEGAWA, Tokyo (JP)

(73) Assignee: RIGAKU CORPORATION, Tokyo (JP)

(21) Appl. No.: 18/075,425

(22) Filed: Dec. 6, 2022

(30) Foreign Application Priority Data
Dec. 10, 2021 (JP) 2021-200965
Oct. 31, 2022 (JP) 2022-174381

(51) Int. Cl. G16B 15/00; G01N 23/2055

(52) U.S. Cl. CPC G16B 15/00; G01N 23/2055; G01N 2223/612

(57) ABSTRACT

Electron density map specifying circuitry is configured to accurately reproduce an electron density map of a macromolecule in a solution having a dynamically fluctuating structure. For example, the electron density map circuitry generates a plurality of electron density maps from a measured X-ray scattering profile acquired by measuring a sample, calculates an index representing a degree of coincidence between an X-ray scattering profile calculated from each of the plurality of electron density maps and the measured X-ray scattering profile, and selects a representative electron density map from the plurality of electron density maps based on the calculated index.

### CROSS-REFERENCE TO RELATED APPLICATIONS

The present application claims priority to Japanese Patent Application No. 2021-200965, filed Dec. 10, 2021, and Japanese Patent Application No. 2022-174381, filed Oct. 31, 2022, the entire contents of each are incorporated herein by reference.

### BACKGROUND

### Field

The present disclosure relates to a three-dimensional electron density map specifying apparatus, system, method, and program for specifying a three-dimensional electron density map of a macromolecule in a solution.

### Description of the Related Art

Observation methods of the biomacromolecule in the solution have been studied further. When X-rays are applied to biomacromolecules that are in a state free to move in a solution, a ring-shaped scattered ray is generated instead of a spot. A technique is known in which such a scattered ray is detected, and a three-dimensional electron density map of a target molecule is obtained from the acquired measured X-ray scattering profile (Non-Patent Document 1).

In the method described in Non-Patent Document 1, the volume of a cube in a real space containing particles is represented by a voxel of a cube discretized in an N×N×N grid, and an electron density map is calculated by iteratively searching for a structural factor based on X-ray scattering data acquired from a sample.

### NON-PATENT DOCUMENTS

Non-patent Document 1: Thomas D Grant, “Ab initio electron density determination directly from solution scattering data”, Nature Methods volume 15, 29 Jan. 2018, pages 191-193

However, in the method described in Non-Patent Document 1, only an electron density map resulting from averaging a plurality of electron density maps is obtained. Therefore, when analyzing the form of a rigid molecule, a reasonable electron density map is calculated. However, in the case of a flexible molecule, the form of dynamic structures is averaged, and the electron density to be originally obtained lacks the characteristics of the molecule and has little mean.

### SUMMARY

The present disclosure has been made in view of such circumstances, and the present disclosure includes an electron density map specifying apparatus, system, method, and a program capable of reproducing an electron density map of a may in a solution having a flexible nature and showing structure with dynamic fluctuation.

(1) The electron density map specifying apparatus of the present disclosure includes an electron density map specifying apparatus for specifying an electron density map of a macromolecule in a solution and comprises an electron density map generating section for generating a plurality of electron density maps from a measured X-ray scattering profile acquired by measuring a sample, an index calculating section for calculating an index representing a degree of coincidence between an X-ray scattering profile calculated from each of the plurality of electron density maps and the measured X-ray scattering profile, and an electron density map selecting section for selecting a representative electron density map from the plurality of electron density maps based on the calculated index.

(2) Further, the electron density map specifying apparatus of the present disclosure further comprises a correlation determining section for generating a first plot of a parameter representing a molecular size with respect to the calculated index for each of the plurality of electron density maps and determining the presence or absence of correlation of the first plot, wherein the electron density map selecting section does not select the representative electron density map when there is no meaningful correlation of the first plot.

(3) Further, the electron density map specifying apparatus of the present disclosure further comprises a trend analyzing section for performing multivariate analysis on the distribution of the first plot when there is no correlation of the first plot, wherein the electron density map generating section generates the plurality of electron density maps by changing a condition when there is a trend in the distribution of the first plot.

(4) Further, in the electron density map specifying apparatus of the present disclosure, the correlation determining section generates an outputable first plot when there is no correlation in the first plot, and the electron density map generating section generates the plurality of electron density maps under a condition based on an instruction from a user.

(5) Further, in the electron density map specifying apparatus of the present disclosure, the electron density map generating section generates each of the plurality of electron density maps one by one in a repetitive process according to a setting.

(6) Further, in the electron density map specifying apparatus of the present disclosure, the electron density map generating section generates each of the plurality of electron density maps at a time in parallel processes according to a setting.

(7) Further, the electron density map specifying apparatus of the present disclosure further comprises a theoretical size calculating section for generating a second plot of a parameter representing a molecular size calculated based on the plurality of electron density maps with respect to each voxel size and calculating a calculated value of a parameter representing a molecular size of a macromolecule in the solution using the second plot, and an actual size calculating section for calculating a parameter representing a molecular size of a may in the solution from the measured X-ray scattering profile as an actual measurement value, wherein the electron density map selecting section does not select the representative electron density map unless a difference between the calculated value and the actual measurement value is within a predetermined range.

(8) Further, the system of the present disclosure comprises an X-ray solution scattering apparatus and the electron density map specifying apparatus according to any one of (1) to (7), wherein the electron density map specifying apparatus specifies an electron density map of a macromolecule in the solution based on an X-ray scattering profile of the macromolecule in the solution measured by the X-ray solution scattering apparatus.

(9) Further, the method of the present disclosure is an electron density map specifying method for specifying an electron density map of a macromolecule in a solution and comprises the steps of generating a plurality of electron density maps from a measured X-ray scattering profile acquired by measuring a sample, calculating an index representing a degree of coincidence between an X-ray scattering profile calculated from each of the plurality of electron density maps and the measured X-ray scattering profile, and selecting a representative electron density map from the plurality of electron density maps based on the calculated index.

(10) Further, the program of the present disclosure is an electron density map specifying program for specifying an electron density map of a macromolecule in a solution and causes a computer to perform the following processes of generating a plurality of electron density maps from a measured X-ray scattering profile acquired by measuring a sample, calculating an index representing a degree of coincidence between an X-ray scattering profile calculated from each of the plurality of electron density maps and the measured X-ray scattering profile, and selecting a representative electron density map from the plurality of electron density maps based on the calculated index.

### BRIEF DESCRIPTION OF THE DRAWINGS

FIG. 1 is a schematic diagram showing the system of the present disclosure.

FIG. 2 is a perspective view showing an X-ray solution scattering apparatus.

FIG. 3 is a block diagram showing the system of the present disclosure.

FIG. 4 is a flowchart showing the operation of the electron density map specifying apparatus of the present disclosure.

FIG. 5 is a graph showing an example of an X-ray scattering profile.

FIG. 6 is a schematic diagram showing the electron density map.

FIG. 7A is a graph showing ideal distributions of χ²-Rg(ind).

FIG. 7B is a graph showing unanalyzable distributions of χ²-Rg(ind).

FIG. 8 is a graph showing a distribution of Rg(ind) with respect to χ² of the examples.

FIG. 9 is a graph showing extrapolation of Rg(sect) with respect to voxel size in the embodiment.

FIG. 10 is a list showing electron density maps and processing results in the example.

FIG. 11A is a front side view showing the selected electron density map. In order to help understand the obtained electron density map, a structural ribbon model obtained separately by X-ray crystal structure analysis is superposed.

FIG. 11B is a plan view showing the selected electron density map, and in order to help understand the obtained electron density map, a structural ribbon model obtained separately by X-ray crystal structure analysis is superposed.

FIG. 11C is a right side view showing the selected electron density map respectively, and in order to help understand the obtained electron density map, a structural ribbon model obtained separately by X-ray crystal structure analysis superposed.

### DETAILED DESCRIPTION

Next, embodiments of the present disclosure are described with reference to the drawings. To facilitate understanding of the description, the same reference numerals are assigned to the same components in the respective drawings, and duplicate descriptions are omitted.

### [Electron Density Map Specific System]

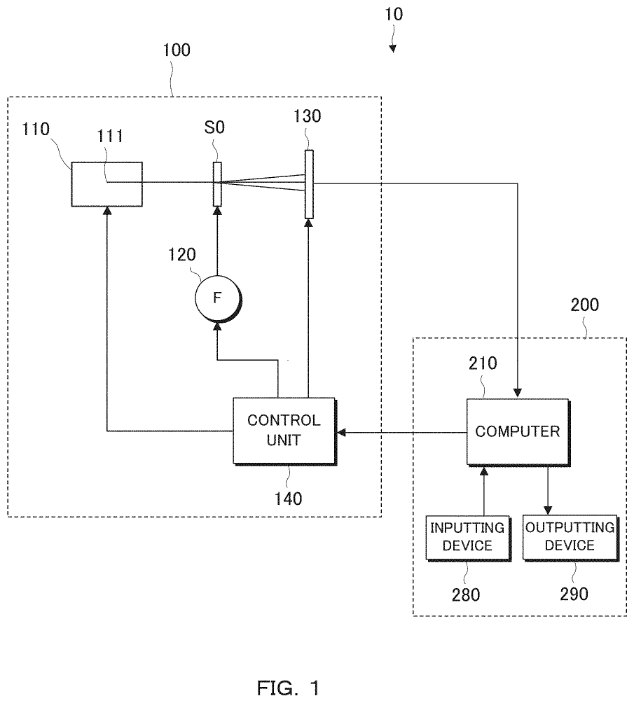

FIG. 1 is a schematic diagram showing the electron density map specifying system 10. The electron density map specifying system 10 comprises an X-ray solution scattering apparatus 100 and an electron density map specifying apparatus 200. The X-ray solution scattering apparatus 100 measures the X-ray solution scattering profile by irradiating the sample S0 with X-rays and detecting the scattered X-rays. The sample S0 is suitable to be macromolecules in solutions, in particular biomacromolecules. The sample S0 may be a pharmaceutical molecule, molecular complex or structure in solution. Further, by using the X-ray solution scattering method, it is possible to visualize an electron density corresponding to an ensemble of the structure of the biomacromolecule which is not observable in the freezing or crystalline state.

The electron density map specifying apparatus 200 comprises a computer 210, an inputting device 280, and an outputting device 290, and controls the operation of the X-ray solution scattering apparatus 100 and acquires measurement data from the X-ray solution scattering apparatus 100 and processes the data.

The X-ray solution scattering apparatus 100 comprises an X-ray generation unit 110, a sample loading mechanism 120, a detector 130, and a control unit 140. The X-ray generation unit 110 has an X-ray source 111 and irradiates the sample S0 with X-rays. The sample loading mechanism 120 delivers a sample solution to an X-ray irradiation position or a sample solution eliminating the macromolecule component. The detector 130 detects X-rays scattered by the sample S0 and transmits the acquired measured data to the computer 210.

The computer 210 is, for example, a PC, and comprises a processor that executes processes, a memory that stores programs and data, a hard disk, and the like. The computer 210 receives user input from an inputting device 280 such as a keyboard or a mouse. Meanwhile, the computer 210 displays a plot, a visualized electron density map, an input screen, and the like on an outputting device 290 such as a display. The computer 210 may be a server device placed on a cloud. In addition, from the viewpoint of processing load, the function for controlling the operation of the X-ray solution scattering apparatus 100 and the function for processing the measured data may be separated, and the control may be executed by a PC placed at the site, and the data processing may be executed by the server device. Additionally, it should be appreciated that the functionality of the elements disclosed herein may be implemented using circuitry or processing circuitry which includes general purpose processors, special purpose processors, integrated circuits, ASICs (“Application Specific Integrated Circuits”), conventional circuitry and/or combinations thereof which are configured or programmed to perform the disclosed functionality. Processors are considered processing circuitry or circuitry as they include transistors and other circuitry therein. The processor may be a programmed processor which executes a program stored in a memory. In the disclosure, the circuitry, sections, devices, units, or means are hardware that carry out or are programmed to perform the recited functionality. The hardware may be any hardware disclosed herein or otherwise known which is programmed or configured to carry out the recited functionality. When the hardware is a processor which may be considered a type of circuitry, the circuitry, sections, devices, means, or units are a combination of hardware and software, the software being used to configure the hardware and/or processor.

### [X-Ray Solution Scattering Apparatus]

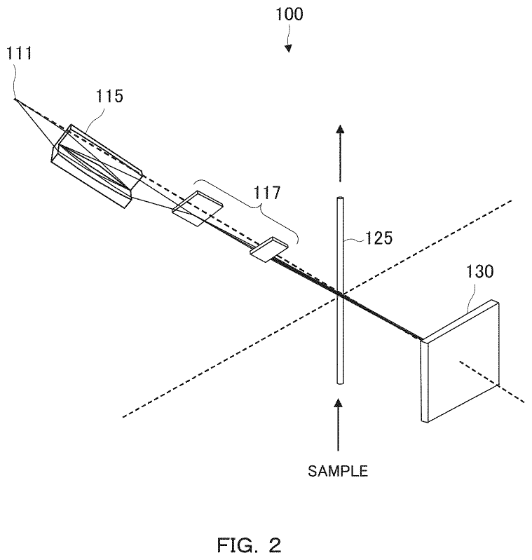

FIG. 2 is a perspective view showing the X-ray solution scattering apparatus 100. The X-ray solution scattering apparatus 100 comprises an X-ray source 111, an optical system 115, a Kratky block 117, a sample holding tube 125, and a detector 130. The X-ray source 111 is a line radiation source or a point radiation source and emits a divergent beam. The optical system 115 is, for example, a KB parallel-type or series-type optical system. A pair of Kratky blocks 117 interact with the x-rays by their respective edges to define one side and the other side of the x-ray beam. Thus, parasitic scattering can be removed from the irradiated X-rays. The sample holding tube 125 delivers and holds 1 μl to 20 μl of the solution sample. The detector 130 detects X-rays scattered by the solution sample.

### [Electron Density Map Specifying Apparatus]

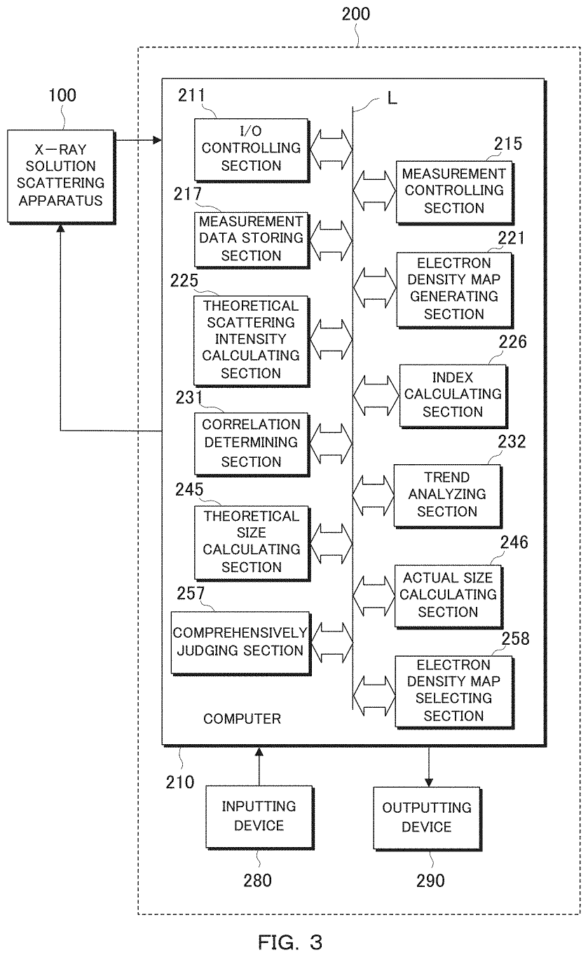

FIG. 3 is a block diagram showing the electron density map specifying system 10. The electron density map specifying apparatus 200 acquires data of the X-ray scattering profile measured by the X-ray solution scattering apparatus 100 and specifies an electron density map of the macromolecule based on the data. The functions of the electron density map specifying apparatus 200 are mainly realized by a computer 210.

The computer 210 comprises an I/O controlling section 211, a measurement controlling section 215, a measurement data storing section 217, an electron density map generating section 221, a theoretical scattering intensity calculating section 225, an index calculating section 226, a correlation determining section 231, a trend analyzing section 232, a theoretical size calculating section 245, an actual size calculating section 246, a comprehensively judging section 257, and an electron density map selecting section 256. Each section can transmit and receive information via the control bus L.

The I/O controlling section 211 receives an input from the inputting device 280 and controls an output to the outputting device 290. The I/O controlling section 211 can receive, for example, an input of a measurement condition or an input of a condition for generating a plurality of electron density maps. Further, the I/O controlling section 211 can output various plots or output an electron density map of the specified macromolecule.

The measurement controlling section 215 controls the operations of the X-ray solution scattering apparatus 100. The controls are for sample delivery, X-ray generation, and sample position and detector movement. The control instruction is transmitted to the control unit 140 in the X-ray solution scattering apparatus 100, and thus each section of the X-ray solution scattering apparatus 100 is controlled.

The measurement data storing section 217 stores measurement data of the X-ray solution scattering profile detected by the X-ray solution scattering apparatus 100. The stored measurement data is used for generating of an electron density map, calculation of an index, and calculation of an actual measurement value of a molecular size of a macromolecule.

The electron density map generating section 221 generates a plurality of electron density maps from the measured X-ray scattering profile acquired by measuring the sample S0. Details of generating of the electron density map are described later.

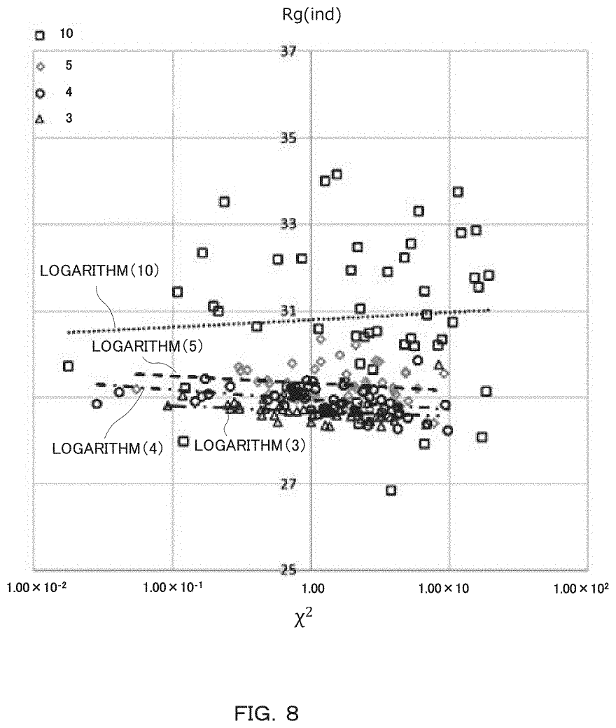

The first plot (see FIG. 8 illustrated later) can be obtained by plotting Rg(ind) values, which indicate the calculated hydrodynamic radius of gyration from an electron density map, to the values of χ² indicating the degree of coincidence among the measured scattering curves and those calculated from the electron density maps. The hydrodynamic radius of gyration Rg of the molecule is an example of a parameter representing the molecular size and may be another parameter representing the molecular size.

The electron density map generating section 221 generates a plurality of electron density maps after changing the condition when the distribution of the first plot has a trend. Thus, it is possible to try to regenerate the electron density map when none of the electron density maps is valid. The electron density map generating section 221 may generate a plurality of electron density maps under a condition based on an instruction from the user. Thus, the electron density map can be tried to be generated by changing the condition when none of the electron density maps are valid.

The electron density map generating section 221 can generate each of the plurality of electron density maps one by one in a repetitive process according to the setting. Thus, it is possible to proceed with the processing by allocating necessary computational resources while reducing unnecessary processing and confirming the validity of the electron density map. Further, the electron density map generating section 221 may generate each of the plurality of electron density maps at one time in parallel processing according to the setting. Thus, the electron density map of the biomacromolecule with high validity can be reproduced in a short time.

The index calculating section 226 calculates an index indicating the degree of coincidence between the X-ray scattering profile calculated from each of the plurality of electron density maps and the measured X-ray scattering profile. Specifically, χ² is an index for which the statistical process has been performed, but the index is not particularly limited as long as it is an index indicating the degree of coincidence with the X-ray scattering profile. In addition to χ², a parameter of a normal distribution or a Poisson distribution, R values, RMS values, and RMD values of an index indicating the degree of coincidence between the structural factors obtained from the measured diffraction data and the structural factors based on the electron density obtained from the analysis, and the like are included. χ² can be calculated as follows.

$$χ2 = {[ \frac{{I}_{exp}^{( i )} ( {q}_{j} ) - {I}_{calc}^{( i )} ( {q}_{j} )}{σ ( {q}_{j} )} ]}^{2} \qquad ( 1 )$$

scattering intensity I_{calc}(q) and actual measurement value Iₑₓₚ(q) σ(q) is a standard deviation of Iₑₓₚ(q)

The correlation determining section 231 generates a first plot by plotting a parameter representing the molecular size with respect to the calculated index for each of the plurality of electron density maps and determines the presence or absence of correlation of the first plot. The correlation determining section 231 generates an outputable first plot and shows the first plot on a display. Thus, the user can visually confirm the presence or absence of correlation in the first plot.

In the case where the repetitive processing is performed, the correlation determining section 231 determines whether or not there is a correlation in the plot for each round of the repetitive processing. Thus, the processing can be proceeded while confirming the validity of the electron density map for each round of the repetitive processing. As a result, the processing can be efficiently performed when the computational resources are limited.

The trend analyzing section 232 performs multivariate analysis on the distribution of the first plot when there is no correlation in the first plot. Examples of the multivariate analysis include MCA(Multiple correspondence analysis) and PCA (principal component analysis). Thus, for example, it is possible to determine the presence or absence of any trend even if there is no correlation. For example, even when there is no correlation in the whole data, it is possible to use various types of data as correlated data by separating a plurality of types of correlated data.

The theoretical size calculating section 245 generates a second plot by plotting a parameter representing the molecular size calculated based on the plurality of electron density maps with respect to voxel sizes. The parameter representing the calculated molecular size is Rg(sect) as an intercept at χ²=1 on the regression line of Rg(ind) as a calculated radius of gyration. Then, the theoretical size calculating section 245 calculates Rg(calc) as a calculated value of a parameter representing the molecular size of the macromolecule in the solution regardless of the voxel size using the second plot.

The actual size calculating section 246 calculates a parameter representing the molecular size of the macromolecule in the solution from the measured X-ray scattering profile as an actual measurement value. Specifically, a Guinier plot can be performed to calculate the Rg(exp) as the actual measurement value of the hydrodynamic radius of gyration of the molecule. Regarding the sample with hydration shells in the solution, one aspect is to treat a value obtained by subtracting a predetermined value for the thickness of the hydration sphere from a value obtained from the Guinier plot as an actual measurement value Rg(exp).

The comprehensively judging section 257 determines whether or not there is no statistically significant difference between the calculated value Rg(calc) of the radius of gyration calculated by the theoretical size calculating section 245 and the actual measurement value Rg(exp) calculated by the actual size calculating section 246. When it is determined that there is no significant difference, a representative electron density map is selected from a plurality of electron density maps. If it is determined that there is a significant difference, the process is terminated.

The electron density map selecting section 258 selects a representative electron density map from the plurality of electron density maps based on the calculated index. Specifically, an electron density map being in accordance with Rg-χ² correlations of the plurality of electron density maps obtained and having a χ² close to 1 is selected as a representative electron density map. Since the representative electron density map is selected based on the index, the representative electron density map of the macromolecule in the solution which has a flexible nature and showing structure with dynamic fluctuation can be reproduced accurately. As a result, biomacromolecules in a solution without any prior information can be accurately visualized.

In one aspect, the electron density map selecting section 258 does not select a representative electron density map when there is no correlation in the first plot. Thus, if none of the electron density maps is valid, specification of the electron density map can be stopped, and unnecessary calculation can be omitted. Then, according to the situation, the electron density map can be generated again, or the other experiments are conducted again for the electron density map(s).

In one aspect, the electron density map selecting section 258 does not select a representative electron density map unless the difference between the calculated value and the actual measurement value is within a predetermined range. Thus, even when the electron density map can be specified, the specification of the electron density map can be stopped when the electron density map is not guaranteed to be valid in terms of the molecular size.

### [Electron Density Map Specifying Method]

### (Entire Method)

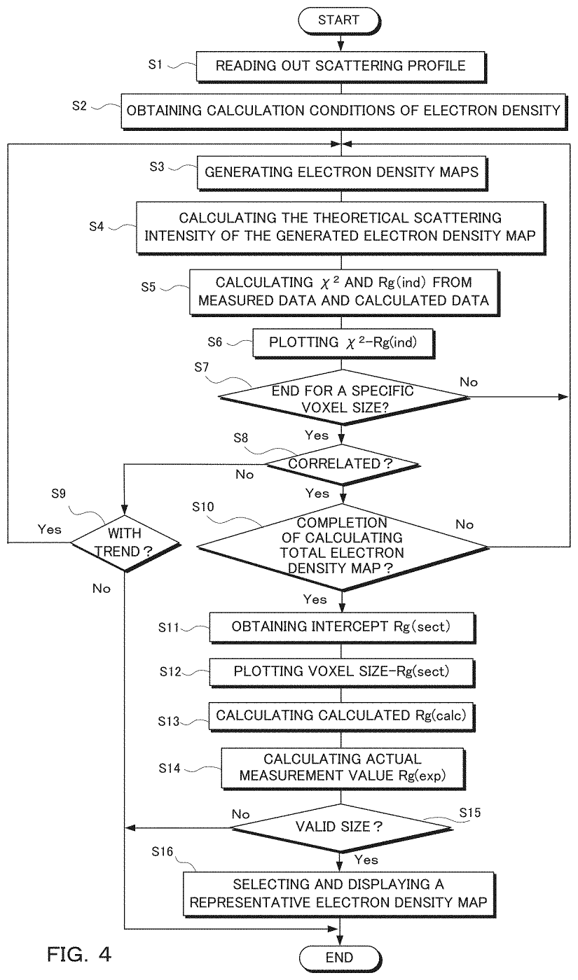

A method of specifying an electron density map of a macromolecule in a solution using the electron density map specifying system 10 configured as described above is described here. FIG. 4 is a flowchart showing the operation of the electron density map specifying apparatus 200. First, the X-ray solution scattering apparatus 100 delivers the solution with the sample S0 to a predetermined position and irradiates the sample S0 with X-rays. The X-ray solution scattering apparatus 100 detects the scattered X-rays and transmits the detected data to the computer 210 as data of an X-ray scattering profile. The computer 210 stores data of the received X-ray scattering profile.

The computer 210 reads out the X-ray scattering profile acquired according to the sample S0 for which the electron density map is to be specified by the user (step S1). Then, generation conditions of the electronic density map, which are specified by the user, such as the boundary value size, the voxel size, and the number of trials for each voxel size are acquired (step S2).

An electron density map obtained from the read-out X-ray scattering profile is generated in accordance with the acquired generation conditions (step S3). Details of generation of the electron density map are described later. Next, a theoretical scattering profile is calculated based on the generated electron density map (step S4). Based on the read-out measured X-ray scattering profile and the calculated theoretical scattering profile, a calculated value Rg(ind) as the particle radius of gyration for each electron density map of the objective molecule and an index χ² representing the degree of coincidence between the measured X-ray scattering profile and the calculated X-ray scattering profile based on an electron density map are calculated (step S5), and a plot of χ²-Rg(ind) is generated (step S6).

Next, it is determined for the repetitive condition whether or not the generation of the electron density map has been tried a predetermined number of times for a particular voxel size (step S7). If it is determined that the trial has not been performed the predetermined number of times, the process returns to step S3. If it is determined that the predetermined number of trials has been performed, the process proceeds to step S8. The predetermined number of times is, for example, 50. In the above-described example, the repetitive processing is performed for a particular voxel size in the step S7, but the repetitive processing may be simply performed a predetermined number of times.

Thus, it is determined whether or not the plotted χ²-Rg(ind) generated for a particular voxel size is correlated (step S8). Details of the correlation determining process are described below. When it is determined that there is no correlation, it is determined whether or not there is any trend in the plot by multivariate analysis or the like (step S9). If it is determined that there is a trend, the process returns to step S3, and the electron density map is generated again by changing the condition. When it is determined that there is no trend, the series of processes is ended without selecting a representative of the electron density maps. When the repetitive processing of the step S7 is completed, the presence or absence of correlations is determined, and the processing with no prospect is terminated, whereby the computational resource can be efficiently used.

On the other hand, if it is determined that there is a correlation in the step S8, it is determined whether or not the condition for terminating the repetition, which the electron density maps are completed to be generated for all of the plurality of voxel sizes, is satisfied (step S10), and if it is determined that the condition is not satisfied, the voxel size is changed and the process returns to the step S3.

In the step S10, when the condition for terminating the repetition is satisfied, a regression line is obtained based on the plotting of χ²-Rg(ind) according to the aggregation of the electron-density maps, and the intercept Rg(sect) at χ²=1 of the regression line is calculated (step S11). Then, a plot of the intercept Rg(sect) with respect to the voxel size is generated (step S12), a regression line is obtained based on the plot, and a calculated value Rg(calc) of the radius of gyration of the molecule is calculated as an extrapolated section where the voxel size is zero in the regression line (step S13). On the other hand, a Guinier plot is generated for the read-out X-ray scattering profile, and an actual measurement value Rg(exp) of the radius of gyration of the molecules is calculated (step S14).

Next, it is determined whether or not the calculated value Rg(calc) is valid by determining whether or not the difference between the calculated value Rg(calc) of the radius of gyration and the actual measurement value Rg(exp) is within a certain range (step S15). At this time, when the sample forms a hydration shells in the solution, one aspect is to treat a numerical value obtained by subtracting a predetermined value for the hydration shell thickness from a value obtained from the Guinier plot as the actual measurement value Rg(exp). In the case that the hydration shell is formed, the predetermined value can be 1.5 Å to 2.0 Å, for example. Alternatively, the thickness of the hydration shells estimated by various calculation methods for the purpose of explicitly calculating the presence of the hydration shells may be used.

In the step S15, if the calculated value Rg(calc) is not determined to be valid, the series of processes is terminated without selecting representative one of the electron density maps. If it is determined that the calculated value Rg(calc) is valid, a representative electron density map is selected among the electron density maps having a χ² close to 1 among the electron density maps according to the series of χ²-Rg correlations, and the selected electron density map is outputted to the outputting device 290 (step S16), and the series of processes is ended.

The representative electron density map is not necessarily single and may be plural. For example, if a plurality of electron density maps in which the plotting of χ²-Rg(ind) has a strong series of correlations and χ² is very close to 1 are obtained, they may be selected as electron density maps corresponding to the respective canonical structure of the dynamic structure ensemble. In this case, it is desirable to select the smallest voxel size among the voxel sizes used for correlation mapping. Or, in this case, it is desirable to select the correlation defined with the smallest voxel value among correlations generated with various voxel values.

In the above example, each of the plurality of electron density maps is generated for each repetition but may be generated at once by parallel processes. Further, a certain number of parallel processes may be repeatedly performed. Which process to be chosen can be determined based on which of the computational resources and the speed of outcome is important.

### Generation of Electron Density Map

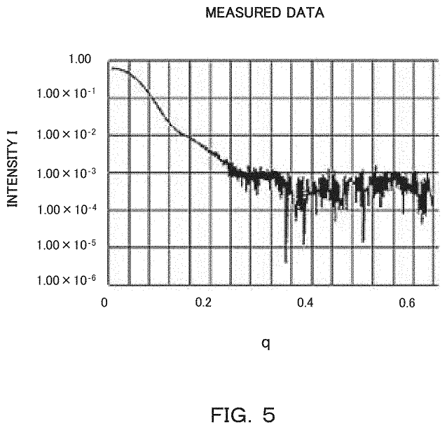

Next, the generation of the electron density map is described here in detail. FIG. 5 is a graph showing an example of an X-ray solution scattering profile. When the biomacromolecules in the solution are irradiated with X-rays, a gradual ring-shaped intensity peak of scattered X-rays is generated in the approximate region of q≤0.7 Å⁻¹. By integrating it in the circumferential direction, an X-ray solution scattering profile as shown in FIG. 5 is obtained. In particular, it is possible to directly visualize the electron density that is meaningful to the actual structure by acquiring and analyzing the scattered intensity data in the q range up to 0.7 Å⁻¹ with high accuracy.

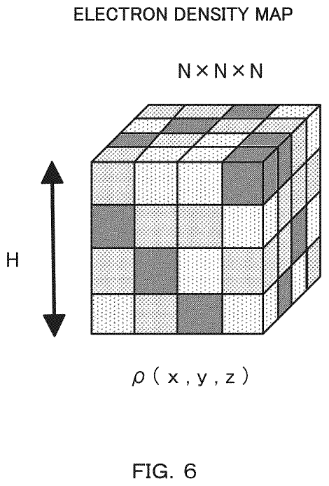

FIG. 6 is a schematic diagram showing the electron density map. In the generation of the electron density map, first, the volume of the cube box with one side H including the macromolecule in the real space is discretized into an N×N×N grid of the voxels of the cube (N=4 in the example shown in FIG. 6). As indicated by the grayscale of each voxel in FIG. 6, the electronic density ρ(x,y,z) of each voxel is randomly given a numerical value in a certain range. Then, the three-dimensional reciprocal lattice space intensities are calculated from the three-dimensional structural factors, and are divided into concentric shells as a function of the magnitude of the scattering vector q.

The three-dimensional scatter intensities are then transformed into a one-dimensional profile and compared to experimental scattering data. Three-dimensional structural factors are scaled to match the experimental data of the concentric shells of each q, and an inverse Fourier transform is performed to generate a new electron density map in real space. The density outside the map is set to zero. new structural factors are obtained by the forward Fourier transformation and this cycle is repeated until convergence. Thus, different electron density maps are generated for each trial, and index χ² and calculated value Rg(ind) are calculated for each of the electron density maps.

### Correlation Determination Process

By plotting the calculated χ² and Rg(ind) for each electron density map and determining whether they are correlated with each other, it is possible to determine the validity of the electron density map with respect to the measured data. Whether or not there is a correlation in the plot can be objectively determined, for example, by using a correlation coefficient. Furthermore, when there is no correlation, by determining whether or not there is a trend in the plot, it is possible to determine whether the data is unlikely to be meaningful or can be meaningful depending on the measurement conditions.

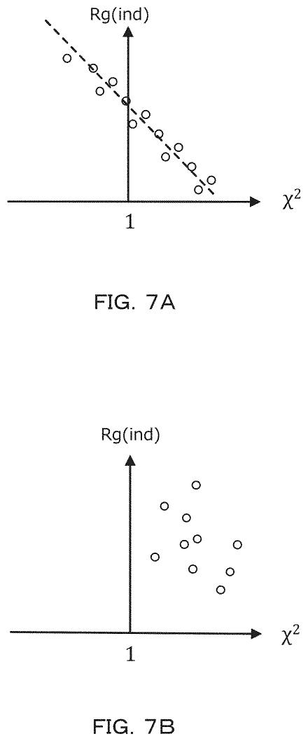

FIGS. 7A and 7B are graphs showing ideal and unanalyzable distributions of χ²-Rg(ind) respectively. In the ideal distribution shown in FIG. 7A, correlations appear in the plot of χ²-Rg(ind), and it is evident that a plurality of the electron density maps are valid. On the other hand, in the distributions which cannot be analyzed shown in FIG. 7B, correlations and tendencies do not appear in the plot of χ²-Rg(ind), and it can be seen that a plurality of the electron-density maps are not valid. If the electron density maps are valid, an electron density map having a χ² within 1 to a predetermined range can be selected as a representative electron density map.

### Example

In practice, electron density maps were specified using samples of biomacromolecules in solutions (human serum-albumin (HSA)). Electron density maps were generated by 50 trials for each voxel size at 10 Å, 5 Å, 4 Å, and 3 Å. Then, χ² and Rg(ind) of the generated electron density maps were calculated. FIG. 8 is a graph showing the distribution of the calculated Rg(ind) of molecular radius of gyration with respect to the index χ² of the examples. The correlation was confirmed for each voxel size at 10 Å, 5 Å, 4 Å, and 3 Å.

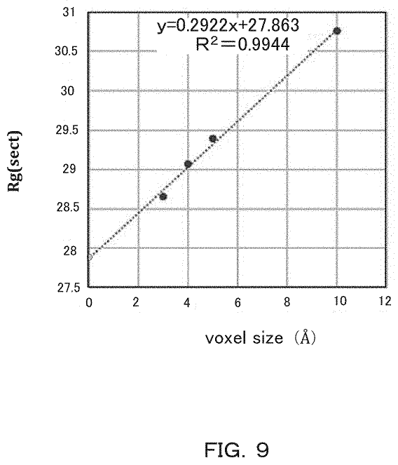

Then, the respective Rg(sect) of the voxel sizes 10 Å, 5 Å, 4 Å, and 3 Å was calculated on the basis of a straight line representing the correlation. FIG. 9 is a graph showing extrapolation of Rg(sect) with respect to voxel size in the embodiment. The voxel sizes and the obtained Rg(sect) were plotted to calculate Rg(calc) for the entire plurality of the electron density maps. These results indicate that the generated electron density maps are valid.

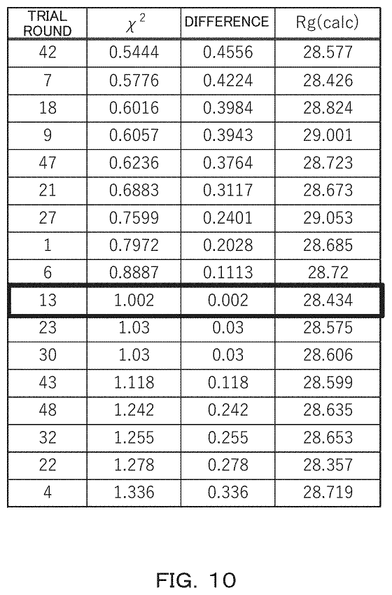

FIG. 10 is a list showing electron density maps and processing results in the example. Since more reasonable electron density maps can be obtained for a smaller voxel size, χ² for trials of a voxel size of 3 Å are arranged in ascending order. According to the obtained list, the electron density map of the thirteenth trial with 0.002 as a difference between χ² and 1 is most valid, so this has been selected as the representative electron density map.

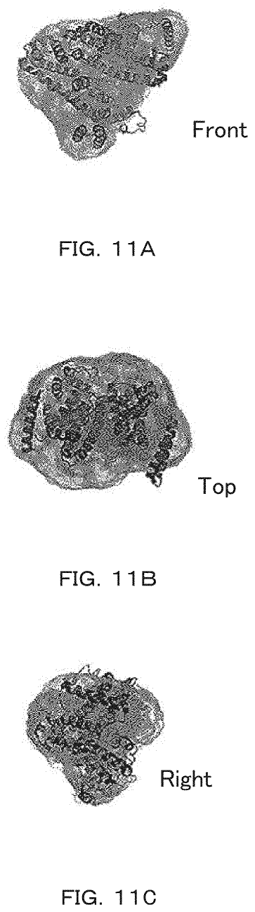

FIGS. 11A to 11C are a front, plan and right side view showing the selected electron density map respectively. As described above, the electron density map in the thirteenth trial for a voxel size of 3 Å has been specified and visualized.

The visualized electron density indicates a shape like a human liver as a whole. This is a good representation of the molecular surface shape obtained from X-ray crystallography of HSA. Further, in FIG. 11A, a depression of about 5 Å and a cylindrical bulge along the depression are observed from the center of the longest side in the bottom right toward the top left. The visualized shape coincides well with the shape obtained from the X-ray structure analysis not only in terms of the overall shape but also in terms of the fine features of the molecular surface structure.

What is claimed is:

1. An electron density map specifying apparatus for specifying an electron density map of a macromolecule in a solution, comprising:
processing circuitry configured to
generate a plurality of electron density maps from a measured X-ray scattering profile acquired by measuring a sample,
calculate an index representing a degree of coincidence between an X-ray scattering profile calculated from each of the plurality of electron density maps and the measured X-ray scattering profile, and
select a representative electron density map from the plurality of electron density maps based on the calculated index.

2. The electron density map specifying apparatus according to claim 1, wherein the processing circuitry is further configured to
generate a first plot of a parameter representing a molecular size with respect to the calculated index for each of the plurality of electron density maps and determine the presence or absence of correlation of the first plot,
wherein the representative electron density map is not selected when there is no correlation of the first plot.

3. The electron density map specifying apparatus according to claim 2, wherein the processing circuitry is further configured to
perform multivariate analysis on the distribution of the first plot when there is no correlation of the first plot, and
generate the plurality of electron density maps by changing a condition when there is a trend in the distribution of the first plot.

4. The electron density map specifying apparatus according to claim 2, wherein the processing circuitry is further configured to
generate an outputable first plot when there is no correlation in the first plot, and generate the plurality of electron density maps under a condition based on an instruction from a user.

5. The electron density map specifying apparatus according to claim 1, wherein the processing circuitry is further configured to
generate each of the plurality of electron density maps one by one in a repetitive process according to a setting.

6. The electron density map specifying apparatus according to claim 1, wherein the processing circuitry is further configured to
generate each of the plurality of electron density maps at a time in parallel processes according to a setting.

7. The electron density map specifying apparatus according to claim 1, wherein the processing circuitry is further configured to
generate a second plot of a parameter representing a molecular size calculated based on the plurality of electron density maps with respect to each voxel size and calculate a calculated value of a parameter representing a molecular size of a macromolecule in the solution using the second plot, and
calculate a parameter representing a molecular size of a macromolecule in the solution from the measured X-ray scattering profile as an actual measurement value,
wherein the representative electron density map is not selected unless a difference between the calculated value and the actual measurement value is within a predetermined range.

8. A system, comprising:
an X-ray solution scatterer that measures an X-ray solution scattering profile; and
electron density map specifying circuitry, the electron density map specifying circuitry being configured to
generate a plurality of electron density maps from a measured X-ray scattering profile acquired by measuring a sample,
calculate an index representing a degree of coincidence between an X-ray scattering profile calculated from each of the plurality of electron density maps and the measured X-ray scattering profile, and
select a representative electron density map from the plurality of electron density maps based on the calculated index,
wherein an electron density map of a macromolecule in the solution is specified based on an X-ray scattering profile of the macromolecule in the solution measured by the X-ray solution scatterer.

9. An electron density map specifying method for specifying an electron density map of a macromolecule in a solution, comprising the steps of:
generating a plurality of electron density maps from a measured X-ray scattering profile acquired by measuring a sample,
calculating an index representing a degree of coincidence between an X-ray scattering profile calculated from each of the plurality of electron density maps and the measured X-ray scattering profile, and
selecting a representative electron density map from the plurality of electron density maps based on the calculated index.

10. A non-transitory computer readable recording medium having recorded thereon a electron density map specifying program for specifying an electron density map of a macromolecule in a solution, the program causing a computer to perform the following processes of:
generating a plurality of electron density maps from a measured X-ray scattering profile acquired by measuring a sample,
calculating an index representing a degree of coincidence between an X-ray scattering profile calculated from each of the plurality of electron density maps and the measured X-ray scattering profile, and
selecting a representative electron density map from the plurality of electron density maps based on the calculated index.

11. The method of claim 9, further comprising:
generating a first plot of a parameter representing a molecular size with respect to the calculated index for each of the plurality of electron density maps and determine the presence or absence of correlation of the first plot,
wherein the representative electron density map is not selected when there is no correlation of the first plot.

12. The method of claim 11, further comprising:
performing multivariate analysis on the distribution of the first plot when there is no correlation of the first plot; and
generating the plurality of electron density maps by changing a condition when there is a trend in the distribution of the first plot.

13. The method of claim 11, further comprising:
generating an outputable first plot when there is no correlation in the first plot, and generate the plurality of electron density maps under a condition based on an instruction from a user.

14. The method of claim 9, further comprising:
generating each of the plurality of electron density maps one by one in a repetitive process according to a setting.

15. The method of claim 9, further comprising:
generating each of the plurality of electron density maps at a time in parallel processes according to a setting.

16. The method of claim 9, further comprising:
generating a second plot of a parameter representing a molecular size calculated based on the plurality of electron density maps with respect to each voxel size and calculate a calculated value of a parameter representing a molecular size of a macromolecule in the solution using the second plot; and
calculating a parameter representing a molecular size of a macromolecule in the solution from the measured X-ray scattering profile as an actual measurement value,
wherein the representative electron density map is not selected unless a difference between the calculated value and the actual measurement value is within a predetermined range.
